(19) United States
(12) Patent Application Publication
(10) Pub. No.: US 2021/0015976 A1
(43) Pub. Date: Jan. 21, 2021

(54) Transcutaneous Intraosseous Devices and Methods for Manufacturing Thereof

(71) Applicants: UNIVERSITE LAVAL, Quebec (CA); LABORATOIRES BODYCAD INC., Quebec (CA)

(72) Inventors: Gaetan LAROCHE, Quebec (CA); Souhaila GHADHAB, Quebec (CA); Andree-Anne GUAY-BEGIN, Levis (CA); Geoffroy RIVET-SABOURIN, Stoneham (CA)

(21) Appl. No.: 16/931,034

(22) Filed: Jul. 16, 2020

Related U.S. Application Data
(60) Provisional application No. 62/876,110, filed on Jul. 19, 2019.

(51) Int. Cl. A61L 27/54; A61L 27/04; A61L 27/06; A61L 27/36; A61L 27/22

(52) U.S. Cl. CPC A61L 27/54; A61L 27/045; A61L 27/06; A61L 2400/06; A61L 27/227; A61L 2400/18; A61L 27/3633

(57) ABSTRACT

Transcutaneous intraosseous devices with enhanced antimicrobial properties, as well as their production processes are described. Particularly, the transcutaneous intraosseous device comprises an intraosseous part and a transcutaneous part, wherein at least a surface of said transcutaneous part is provided with at least one adhesion or proliferation agent and at least one antimicrobial agent. For instance, the transcutaneous intraosseous device is intraosseous transcutaneous amputation prosthesis.

### RELATED APPLICATION

This application claims priority under applicable laws to U.S. provisional application No. 62/876.110 filed on Jul. 19, 2019, the content of which is incorporated herein by reference in its entirety for all purposes.

### TECHNICAL FIELD

The technical field generally relates to transcutaneous intraosseous devices, and more particularly, to intraosseous transcutaneous implants and manufacturing processes thereof.

### BACKGROUND

Commonly used external prostheses for patients who have had limbs amputated (such as socket interface prostheses) are associated to significant disadvantages, for example, excessive pressure areas on soft tissue (e.g. skin), poor fit, discomfort, unnatural gait and limited range of movement.

An integrated prosthetic (intraosseous transcutaneous amputation prosthesis (ITAP)) can address several significant problems associated with common external prostheses. Advantageously, ITAPs are designed as a single-component system implanted in a single surgery. An ITAP allows to anchor a prosthetic limb to an implant inserted into the bone of a stump via a skin-penetrating abutment. An ITAP therefore provides a means to directly attach a prosthetic limb to the skeleton of an amputee. For instance, a prosthesis with a direct connection to the bone can significantly reduce unwanted prosthetic physical interaction with soft tissue and allows mechanical forces to be directly transferred to the skeleton. An ITAP can, for example, improve the comfort, control and fit of a prosthetic limb.

However, the use of intraosseous transcutaneous orthopaedic implants in amputees is associated with incidences of implant-related complications due to poor implant integration, failure to achieve a tight seal between the soft tissue, and the lower implant extremity, inflammation, mechanical instability or infections. These implant-related complications can prolong patient care, cause pain, cause implant failure, and can ultimately lead to removal or replacement surgery. For example, the failure to achieve an adequate seal between the soft tissue and the implant can lead to epithelial downgrowth (i.e., the migration of epithelial cells downwards and parallel to the side of the implant). Epithelial downgrowth can lead to marsupialisation (i.e., the formation of a deep pocket) of the soft tissues thereby creating a route for microorganisms (e.g. bacteria) to enter the underlying soft tissues and to other conditions that are favorable to bacterial proliferation.

Accordingly, there is a need for orthopaedic implants that overcome one or more of the disadvantages encountered with conventional prostheses.

### SUMMARY

According to a first aspect, the present technology relates to a transcutaneous intraosseous device comprising an intraosseous part and a transcutaneous part, wherein at least a surface of said transcutaneous part is provided with at least one adhesion or proliferation agent and at least one antimicrobial agent.

In one embodiment, the transcutaneous intraosseous device is an intraosseous transcutaneous implant used to anchor a prosthetic limb or a dental prothesis.

In another embodiment, the transcutaneous intraosseous device is an intraosseous transcutaneous amputation prosthesis (ITAP).

In another embodiment, the surface of said transcutaneous part has a roughness of less than about 1.1 μm, or less than about 1.0 μm, or less than about 0.9 μm, or less than about 0.8 μm, or less than about 0.7 μm, or less than about 0.6 μm, or less than about 0.5 μm, or less than about 0.4 μm, or less than about 0.3 μm, or less than about 0.2 μm.

In another embodiment, the surface of said transcutaneous part has a roughness in the range of from about 0.2 μm to about 0.5 μm.

In another embodiment, the transcutaneous intraosseous device comprises at least one biomaterial. For instance, the biomaterial is a metal or a metal alloy. For example, the metal or metal alloy is at least one of a stainless steel, a titanium-based material and a cobalt-based material. In one example, the stainless steel is ASTM F316L stainless steel. In another example, the cobalt-based material is a cobalt—chromium (Co—Cr) alloy.

In another example, the titanium-based material is a titanium alloy. In one variant of interest, the titanium alloy is Ti-6Al-4V or Ti-6Al-4V extra low interstitial (Ti-6Al-4V ELI).

In another embodiment, the at least one adhesion or proliferation agent is an extracellular macromolecule. For instance, the extracellular macromolecule is a protein extracted from the extracellular matrix. For example, the protein extracted from the extracellular matrix is an adhesion protein. In one example, the adhesion protein is fibronectin or laminin. In one variant of interest, the adhesion protein is fibronectin.

In another embodiment, the at least one adhesion or proliferation agent comprises a cell adhesion peptide extracted from the extracellular matrix. In one example, the cell adhesion peptide extracted from the extracellular matrix is at least one of Arg-Gly-Asp (RGD), Lys-Arg-Gly-Asp (KRGD), Tyr-Ile-Gly-Ser-Arg (YIGSR) and Lys-Tyr-Ile-Gly-Ser-Arg (KYIGSR). In one variant of interest, the cell adhesion peptide extracted from the extracellular matrix is RGD. In another variant of interest, the cell adhesion peptide extracted from the extracellular matrix is KRGD. In another variant of interest, the cell adhesion peptide extracted from the extracellular matrix is YIGSR. In another variant of interest, the cell adhesion peptide extracted from the extracellular matrix is KYIGSR.

In another embodiment, the at least one antimicrobial agent is at least one of an antimicrobial peptide and an antibiotic. In one example, the antimicrobial peptide is a magainin. In one variant of interest, the magainin is magainin 1 or magainin 2. In another variant of interest, the magainin is magainin 2.

In another embodiment, the antibiotic is at least one of gentamicin, vancomycin, and cefotaxime. In one variant of interest, the antibiotic is cefotaxime.

According to another aspect, the present technology relates to an intraosseous transcutaneous amputation prosthesis (ITAP) comprising an intraosseous part and a transcutaneous part, wherein at least a surface of said transcutaneous part is provided with at least one adhesion or proliferation agent and at least one antimicrobial agent.

In one embodiment, the surface of said transcutaneous part has a roughness of less than about 1.1 μm, or less than about 1.0 μm, or less than about 0.9 μm, or less than about 0.8 μm, or less than about 0.7 μm, or less than about 0.6 μm, or less than about 0.5 μm, or less than about 0.4 μm, or less than about 0.3 μm, or less than about 0.2 μm.

In another embodiment, the ITAP comprises at least one biomaterial. For instance, the biomaterial is a metal or a metal alloy. For example, the metal or metal alloy is at least one of a stainless steel, a titanium-based material and a cobalt-based material. In one example, the titanium-based material is a titanium alloy. In one variant of interest, the titanium alloy is Ti-6Al-4V or Ti-6Al-4V ELI.

In another embodiment, the at least one adhesion or proliferation agent is an extracellular macromolecule. For instance, the extracellular macromolecule is a protein extracted from the extracellular matrix. For example, the protein extracted from the extracellular matrix is an adhesion protein. In one variant of interest, the adhesion protein is fibronectin or laminin. In another variant of interest, the adhesion protein is fibronectin.

In another embodiment, the at least one adhesion or proliferation agent comprises a cell adhesion peptide extracted from the extracellular matrix. In one example, the cell adhesion peptide extracted from the extracellular matrix is at least one of RGD, KRGD, YIGSR and KYIGSR. In one variant of interest, the cell adhesion peptide extracted from the extracellular matrix is RGD. In another variant of interest, the cell adhesion peptide extracted from the extracellular matrix is KRGD. In another variant of interest, the cell adhesion peptide extracted from the extracellular matrix is YIGSR. In another variant of interest, the cell adhesion peptide extracted from the extracellular matrix is KYIGSR.

In another embodiment, the at least one antimicrobial agent is at least one of an antimicrobial peptide and an antibiotic. In one example, the antimicrobial peptide is a magainin. In one variant of interest, the magainin is magainin 1 or magainin 2. In another variant of interest, the magainin is magainin 2.

In another embodiment, the antibiotic is at least one of gentamicin, vancomycin, and cefotaxime. In one variant of interest, the antibiotic is cefotaxime.

According to another aspect, the present technology relates to a process for producing a transcutaneous intraosseous device as defined herein, comprising the following steps:
immobilizing at least one adhesion or proliferation agent onto the surface of the transcutaneous part; and
immobilizing at least one antimicrobial agent onto the surface of the transcutaneous part.

In one embodiment, the process further comprises polishing the surface of the transcutaneous part prior to the immobilizing steps. In one example, the polishing is performed by mechanical polishing using abrasive papers.

In another embodiment, the process further comprises cleaning the surface of the transcutaneous part prior to the immobilizing steps, and if present, after the polishing step. In one example, the cleaning is carried out in an ultrasonic bath. In another example, the cleaning is performed using at least one solvent and/or a mild detergent.

In another embodiment, the process further comprises a surface pre-functionalization or activation step prior to the immobilizing steps.

In another embodiment, the surface pre-functionalization or activation step comprises generating at least one free surface reactive group.

In another embodiment, the surface pre-functionalization or activation step is carried out using an activating agent and is performed by a wet-chemistry functionalization process or a plasma functionalization technique.

In another embodiment, the free surface reactive group comprises at least one of a hydrocarbon-containing group, an oxygen-containing group, a nitrogen-containing group, a phosphorous-containing group, and a sulfur-containing group.

In another embodiment, the free surface reactive group comprises at least one of hydroxyl groups (—OH), amine groups (—NH₂), carboxyl groups (—COOH), and thiol groups (—SH). In one variant of interest, the free surface reactive group comprises hydroxyl groups (—OH).

In another embodiment, the activating agent is at least one of a sodium hydroxide solution, a nitric acid solution, and a piranha solution. In one variant of interest, the activating agent is a sodium hydroxide solution.

In another embodiment, immobilizing the at least one adhesion or proliferation agent and the at least one antimicrobial agent, independently in each occurrence, involves a covalent approach or a non-covalent approach. In one variant of interest, the non-covalent approach is adsorption. In another variant of interest, the covalent approach is at least one of self-assembly of monolayers immobilization, covalent bonding, and covalent grafting.

In another embodiment, immobilizing the at least one adhesion or proliferation agent and/or the at least one antimicrobial agent is performed directly on the surface.

In another embodiment, the process further comprises immobilizing at least one bifunctional molecule onto the surface of the transcutaneous part.

In another embodiment, the at least one bifunctional molecule comprises identical or different reactive groups on a first end and a second end of a spacer arm.

In another embodiment, the spacer arm is an alkyl chain comprising from 10 to 18 carbon atoms. In one variant of interest, the spacer arm is an alkyl chain comprising 16 carbon atoms.

In another embodiment, immobilizing the at least one bifunctional molecule is performed by covalently binding the first end of the space arm onto the surface.

In another embodiment, immobilizing the at least one adhesion or proliferation agent and the at least one antimicrobial agent is performed by covalently binding the second end of the space arm to the at least one adhesion or proliferation agent and/or the at least one antimicrobial agent.

In another embodiment, the at least one bifunctional molecule is at least one of glutaric anhydride, cis-aconitic anhydride, dopamine, polydopamine, and a phosphonate-containing bifunctional molecule. In one variant of interest, the at least one bifunctional molecule is a phosphonate-containing bifunctional molecule. In another variant of interest, the at least one bifunctional molecule is dopamine or polydopamine.

In another embodiment, the at least one bifunctional molecule is the at least one first bifunctional molecule and the process further comprises immobilizing at least one second bifunctional molecule.

In another embodiment, immobilizing the at least one adhesion or proliferation agent and/or the at least one antimicrobial agent is performed by covalently binding a second end of a space arm to the at least one second bifunctional molecule, to the at least one adhesion or proliferation agent, and/or the at least to one antimicrobial agent, covalently binding a first end of the spacer arm of the at least one second bifunctional molecule to the second end of the spacer arm of the at least one first bifunctional molecule and covalently binding the first end of the spacer arm of the at least one first bifunctional molecule onto the surface.

In another example, the at least one first bifunctional molecule is dopamine or polydopamine and the at least one second bifunctional molecule is glutaric anhydride.

In another embodiment, the process further comprises activating the at least one bifunctional molecule.

In another embodiment, the process further comprises activating the at least one second bifunctional molecule.

In another embodiment, activating is performed by using a cross-linking agent. In one variant of interest, the cross-linking agent is 1-ethyl-3-(3-dimethylaminopropyl)-carbodiimide (EDC).

### BRIEF DESCRIPTION OF DRAWINGS

FIG. 1 is a graph of enzyme-linked immunosorbent assay (ELISA) results for fibronectin-modified Ti 6Al-4V ELI discs as prepared in Examples 1 (a) to (c), the assay was carried out as described in Example 1 (d). The results are presented for Ads-FN-modified Ti 6Al-4V ELI, Dop-FN-modified Ti 6Al-4V ELI, and Phos-FN-modified Ti 6Al-4V ELI discs.

FIG. 2 is a graph of cell adhesion assay results for an uncoated Ti 6Al-4V ELI disc, and fibronectin-modified Ti 6Al-4V ELI discs as prepared in Examples 1 (a) to (c), the assay was carried out as described in Example 1 (d).

FIG. 3 is a graph of resazurin cell viability assay results for peptide-modified Ti 6Al-4V ELI discs, as prepared in Examples 2 (a) and (b), the assay was performed as described in Example 2 (d). The results are presented for an uncoated Ti 6Al-4V ELI disc, a KRGDS-modified Ti 6Al-4V ELI disc and a KYIGSR-modified Ti 6Al-4V ELI disc, after 1 day (dot filling pattern), 4 days (diagonal line brick filling pattern), and 7 days (horizontal brick filling pattern).

FIG. 4 is a profilometry image of a polished Ti6Al4V ELI disc as prepared in Examples 3 (a) and (b), the scan was carried out as described in Example 3 (c).

FIG. 5 is a graph of contact angle measurements recoded for at every step of the process as defined in Examples 3 (a) and (b). The contact angle measurements were carried out as described in Example 3 (c).

FIG. 6 displays the high-resolution XPS spectra of the carbon region (C1s) in (a) for Ti-Dop-GA; and in (b) for Ti-Dop-GA-FN, as described in Example 3 (c). The peaks were deconvoluted, and the peak labelling results are presented for C—C and C—H bonds, C—O and C—N bonds, and N—C═O bonds, as indicated by the arrows on the spectra.

FIG. 7 displays the high-resolution XPS C1s spectra obtained in (a) for Ti-Phos; and in (b) for Ti-Phos-FN, as described in Example 3 (c). The peaks were deconvoluted and labelled. The results are presented for C—O , C—P and C—N bonds, N—C═O bonds, and for C—C and C—H bonds as indicated by the arrows on the spectra.

FIG. 8 is a schematic illustration of an ELISA process steps, as described in Example 3 (c).

FIG. 9 include graphs of the ELISA as described in Example 3 (c) performed on modified surfaces where fibronectin was adsorbed or grafted via a phosphonate or a dopamine bifunctional biomolecule. FIG. 9(a) present the result for the use of polyclonal (left bar) and monoclonal (right bar) antibodies; and FIG. 9(b) present the evaluation of monoclonal-to-polyclonal antibodies ratios.

FIG. 10 is a graph of the atomic surface concentrations (atomic %) of carbon, oxygen and nitrogen species (C, O and N) obtained by XPS before and after the grafting of a peptide via a dopamine bifunctional biomolecule, as described in Example 4 (c).

FIG. 11 displays high-resolution XPS spectra after each of the surface modification steps, as described in Example 4 (c). The peaks were deconvoluted and labelled. The results are presented for C—O and C—N bonds, CONH bonds, and for C—C and C—H bonds as indicated by the arrows on the spectra.

FIG. 12 is a graph of the atomic surface concentrations (atomic %) of carbon, oxygen, titanium, nitrogen and phosphorus species (C, O, Ti, N and P) obtained by XPS after surface modification by phosphonates and peptide grafting, as described in Example 4 (c).

FIG. 13 displays high-resolution XPS spectra after each of the surface modification steps, as described in Example 4 (c). The peaks were deconvoluted and labelled. The results are presented for C—O, C—P and C—N bonds, CONH bonds, and for C—C and C—H bonds as indicated by the arrows on the spectra.

### DETAILED DESCRIPTION

The following detailed description and examples are illustrative and should not be interpreted as further limiting the scope of the invention. On the contrary, it is intended to cover all alternatives, modifications and equivalents as can be included as defined by the present description. The objects, advantages and other features of the present techniques will become more apparent and be better understood upon reading of the following non-restrictive description, given with reference to the accompanying drawings.

All technical and scientific terms and expressions used herein have the same definitions as those commonly understood by the person skilled in the art when relating to the present technology. The definition of some terms and expressions used herein is nevertheless provided below for clarity purposes.

When the term “about” is used herein, it means approximately, in the region of or around. When the term “about” is used in relation to a numerical value, it modifies it; for example, by a variation of 10% above and below its nominal value. This term can also take into account the rounding of a number or the probability of random errors in experimental measurements, for instance, due to equipment limitations.

When a range of values is mentioned herein, the lower and upper limits of the range are, unless otherwise indicated, always included in the definition. When a range of values is mentioned in the present application, for instance, a composition range, a concentration range, a size range, or a porosity range, then all intermediate ranges and subranges, as well as individual values included in the ranges, are intended to be included.

It is worth mentioning that throughout the following description when the article “a” is used to introduce an element, it does not have the meaning of “only one” and rather means “one or more”. It is to be understood that where the specification states that a step, component, feature, or characteristic “may”, “might”, “can” or “could” be included, that particular component, feature or characteristic is not required to be included in all alternatives. When the term “comprising” or its equivalent terms “including” or “having” are used herein, it does not exclude other elements. For the purposes of the present invention, the expression “consisting of” is considered to be a preferred embodiment of the term “comprising”. If a group is defined hereinafter to include at least a certain number of embodiments, it is also to be understood to disclose a group, which preferably consists only of these embodiments.

The term “roughness” or its equivalent expression “surface roughness” refer to the profile height deviations from the mean line (i.e., deviation in the direction of the normal vector of a surface). For instance, the roughness can be quantified by a surface-profile measurement made with a profilometer.

The expression “transcutaneous intraosseous device” refers to devices comprising at least an intraosseous part adapted for implantation in a bone and a transcutaneous part.

The term “biomaterial” can be used when referring to natural or synthetic nonviable materials and can include multiple components. For instance, when used in relation to a medical device, or when interacting with and/or providing internal support to biological systems; for instance, when used in joint replacements, dental implants, orthopaedic fixations, intraosseous transcutaneous orthopaedic implants, and stents. Also, the biomaterial can be used in medical applications to enhance, repair, or replace a natural function of a body part. Biomaterials can include a metal, a metal alloy, a polymer, a ceramic, or a composite material. Non-limiting examples of biomaterials used in orthopaedic applications include titanium, titanium alloys, cobalt—chromium alloys, stainless steel, hydroxyapatite, alumina, carbon, polyethylene, poly(methyl methacrylate) (PMMA), and polytetrafluoroethylene (PTFE).

When the term “prosthesis” is used herein, it refers to a device that replaces a limb, organ, or tissue of the body. In one variant of interest, it refers to a device that replaces a limb. The plural form of the term, meaning “prostheses”, can also be used for clarity purposes in the present application.

A biomaterial, an antimicrobial agent, an antibiotic, an antimicrobial peptide, an adhesion or proliferation agent, an adhesion protein, a cell adhesion peptide, a synthesis method, an analytical method, an assay method, or a biological method other than those specifically exemplified can be employed in the practice of the invention without resorting to undue experimentation. All art-known functional equivalents, of any such materials and methods are intended to be included in this invention.

As mentioned above, the success of transcutaneous intraosseous devices is significantly limited by microbial infections that can occur via a breach in the soft tissue. Inadequate soft tissue adhesion to the transcutaneous intraosseous device can lead to bacterial infections. For instance, Staphylococcus aureus, Staphylococcus epidermidis, Escherichia coli, and Pseudomonas aeruginosa are commonly responsible for implant-related infections.

Various techniques described herein relate to the prevention or reduction of infections associated with transcutaneous intraosseous devices. A surface of a transcutaneous part of said transcutaneous intraosseous device is described, the surface being provided with at least one adhesion or proliferation agent and at least one antimicrobial agent. Also described are transcutaneous intraosseous devices having an antimicrobial effect.

According to a first aspect, the present technology thus relates to a transcutaneous intraosseous device comprising an intraosseous part and a transcutaneous part, wherein at least a surface of said transcutaneous part is provided with at least one adhesion or proliferation agent and at least one antimicrobial agent.

In some examples, the transcutaneous intraosseous device is an orthopaedic osseointegrated transcutaneous implant or a dental implant. For instance, the transcutaneous intraosseous device can be an intraosseous transcutaneous implant used to anchor a prosthetic limb such as an intraosseous transcutaneous amputation prosthesis (ITAP). Alternatively, the transcutaneous intraosseous device can be a dental implant used as an orthodontic anchor to support a dental prothesis such as a crown, a bridge, a denture, or a facial prosthesis. In one variant of interest, the transcutaneous intraosseous device is an ITAP, the intraosseous part is a bone anchor configured for insertion into the bone of a stump and the transcutaneous part is a skin-penetrating abutment configured to anchor an external prosthetic device to the bone anchor.

In some examples, the surface of said transcutaneous part can have a roughness of less than about 1.1 μm, or less than about 1.0 μm, or less than about 0.9 μm, or less than about 0.8 μm, or less than about 0.7 μm, or less than about 0.6 μm, or less than about 0.5 μm, or less than about 0.4 μm, or less than about 0.3 μm, or less than about 0.2 μm. In some non-limitative embodiments, the roughness of the surface of said transcutaneous is in the range of from about 0.2 μm to about 0.5 μm, limits included.

In some examples, the transcutaneous intraosseous device can be made with at least one biomaterial, as long as at least a surface of said transcutaneous part is provided with at least one adhesion or proliferation agent and at least one antimicrobial agent. For example, the biomaterial can be selected for its functional performances, biocompatibility, bioactivity, mechanical properties (e.g. hardness, tensile strength, Young's modulus and elongation), resistance to infection, corrosion resistance, wear resistance, reduced toxic emission (e.g. release of metal ions), stability within the implantation site, or sterilizability. For example, the biomaterial can be substantially non-toxic and should cause little to no inflammatory and/or allergic reaction.

In one variant of interest, the biomaterial can include a metal or a metal alloy. For example, the biomaterial can include steel, a titanium-based material, or a cobalt-based material. Any known compatible biomaterial is contemplated, for example, compatible biomaterial material can include surface functional groups such as hydroxyl groups (—OH groups), amine groups (—NH₂), carboxyl groups (—COOH), and thiol groups (—SH). For instance, the biomaterial can include stainless steel (e.g. ASTM F316L stainless steel), cobalt-chromium (Co—Cr) alloys, titanium, or titanium alloys (e.g. Ti-6Al-4V extra low interstitial (Ti-6Al-4V ELI) or Ti-6Al-4V).

According to one example, the biomaterial can include stainless steel. For example, ASTM F316L stainless steel can be selected for its availability, its substantially good biocompatibility, and its relatively low cost.

According to another example, the biomaterial includes a cobalt-based material such as a Co—Cr alloy. For example, the Co—Cr alloy can be selected for its substantially high wear resistance, fatigue resistance, corrosion resistance (compared to stainless steel) or for its substantially good mechanical properties and/or biocompatibility. However, the surface of Co—Cr alloys is prone to corrosion which can lead to the formation of a layer of fibrous tissue between the implant and the soft tissue and thereby can prevent the osseointegration of the implant. The Young's modulus of Co—Cr alloys is substantially high (i.e. about 600 MPA) and can prevent the transmission of mechanical stimuli to the surrounding bone tissue, thereby causing bone resorption. Furthermore, some Co—Cr alloys include substantially toxic elements and thus increase the risk for implant-related complications, due to a possible toxicity. For example, their toxicity can be caused by the release of Ni ions.

According to another example, the biomaterial includes a titanium-based material such as titanium or a titanium alloy. For example, titanium and its alloys provide a substantially high corrosion resistance, substantially good machinability and excellent mechanical properties. Non-limiting examples of titanium alloys include alpha-beta titanium alloys such as Ti-6Al-4V and Ti 6Al-4V ELI. For example, titanium alloys such as Ti-6Al-4V and Ti 6Al-4V ELI, can be preferred to titanium due to their significantly superior mechanical properties. For instance, Ti 6Al-4V has a significantly superior fatigue endurance limit compared to titanium. Ti 6Al-4V ELI is similar to Ti 6Al-4V but has lower inclusions of iron and interstitial elements such as carbon, nitrogen and oxygen. For example, the Ti-6Al-4V can contain about 6 wt. % aluminum, about 4 wt. % vanadium, about 0.25 wt. % iron, and about 0.2 wt. % oxygen. For example, the Ti-6Al-4V ELI can contain about 6 wt. % aluminium, about 4 wt. % vanadium, and about 0.13 wt. % oxygen. For instance, the chemical composition of Ti-6Al-4V and Ti 6Al-4V ELI can be as presented in Table 1.

TABLE 1
Chemical composition of Ti—6Al—4V and Ti 6Al—4V ELI.

| | Composition (wt. %) | |
|---|---|---|
| Elements | Ti—6Al—4V ELI | Ti—6Al—4V |
| N | <0.05 | <0.05 |
| C | <0.08 | <0.10 |
| H | <0.0125 | <0.015 |
| Fe | <0.25 | <0.30 |
| O | <0.13 | <0.20 |
| Al | 5.50-6.50 | 5.50-6.75 |
| V | 3.50-4.5 | 3.50-4.5 |
| Ti | Remainder | Remainder |

In one variant of interest, the biomaterial is a titanium-based material such as titanium or a titanium alloy. For instance, the biomaterial is a titanium alloy, such as Ti-6Al-4V or Ti 6Al-4V ELI.

In some example, the biomaterial can further optionally include impurities (i.e., elements or components) that could be included in smaller amounts. For example, the biomaterial can be substantially free of impurities. For instance, the biomaterial can include less than 1 wt. %, less than 0.5 wt. %, less than 0.4 wt. %, less than 0.3 wt. %, less than 0.2 wt. %, less than 0.1 wt. %, less than 0.05 wt. %, or less than 0.01 wt. % of impurities including N, C, H, Fe and O.

In some examples, the at least one adhesion or proliferation agent and/or the at least one antimicrobial agent can be applied to at least a surface of said transcutaneous part. For example, the at least one adhesion or proliferation agent and/or the at least one antimicrobial agent can be applied to at least a surface at the interface between the stump tissue and the transcutaneous intraosseous device (i.e., skin-implant interface). For instance, the purpose of applying the at least one adhesion or proliferation agent and/or the at least one antimicrobial agent to the surface is functional. For instance, the at least one adhesion or proliferation agent and the at least one antimicrobial agent are provided or applied to the surface in order to provide two or more functional properties that are likely to completely prevent or substantially reduce the colonization of microorganisms (e.g. bacteria). For example, the at least one adhesion or proliferation agent and the at least one antimicrobial agent are provided or applied to promote cell adhesion or proliferation on the surface (passive function) and to provide the surface with antimicrobial (e.g. antibacterial) properties (active function).

For instance, the at least one adhesion or proliferation agent and/or the at least one antimicrobial agent can form a coating on the surface of said transcutaneous part. For example, the at least one adhesion or proliferation agent and/or the at least one antimicrobial agent can completely cover the surface of said transcutaneous part or can only cover parts of the surface of said transcutaneous part. For instance, the at least one adhesion or proliferation agent and/or the at least one antimicrobial agent can form a coating layer or film onto the surface of said transcutaneous part.

In some examples, the adhesion or proliferation agent can promote at least one of the following: cell proliferation, attachment, migration and spreading on the surface of the transcutaneous part. For instance, the adhesion or proliferation agent can reduce epithelial downgrowth, improve epithelial attachment and/or improve dermal attachment. For example, the cells can be skin cells such as epidermal keratinocytes or dermal fibroblasts. The adhesion or proliferation agent can contribute to the formation of a substantially adequate seal between the soft tissue and the transcutaneous part of the transcutaneous intraosseous device and can provide a substantially effective barrier against infection. For example, the adhesion or proliferation agent can be selected for its compatibility with the various elements of the transcutaneous intraosseous device and/or of the body (e.g. human or animal body). Any known compatible adhesion or proliferation agent is contemplated.

In some examples, the adhesion or proliferation agent can be an extracellular macromolecule such as a protein extracted from the extracellular matrix (ECM). For instance, the adhesion or proliferation agent can include an adhesion protein, for example, an adhesion protein extracted from the ECM (e.g. fibronectin, laminin, vitronectin, or collagen). In one alternative, the adhesion or proliferation agent can include a cell adhesion peptide extracted from the proteins of the ECM (e.g. Arg-Gly-Asp (RGD), RGD-containing peptides (e.g. Lys-Arg-Gly-Asp (KRGD), Tyr-Ile-Gly-Ser-Arg (YIGSR) and YIGSR-containing peptides (e.g. Lys-Tyr-Ile-Gly-Ser-Arg (KYIGSR)).

Non-limiting examples of adhesion or proliferation agents include fibronectin, laminin (e.g. laminin 332), RGD, RGD-containing peptides, KRGD, YIGSR, YIGSR-containing peptides, KYIGSR, vitronectin, hydroxyapatite, fibronectin-functionalised hydroxyapatite (HAFN), fibronectin-functionalised hydroxyapatite with silver (HAAgFN), diamond-like carbon (DLC), and combinations thereof. In one variant of interest, the adhesion or proliferation agents include at least one of fibronectin, RGD, KRGDS, YIGSR, or KYIGSR.

In some examples, the antimicrobial agent can prevent, significantly reduce, or treat infections such as bacterial infections. The antimicrobial agent can, for example, inhibit the growth of bacteria. For example, the surface said transcutaneous part can be provided with at least one antimicrobial agent that is effective against gram-positive bacteria, gram-negative bacteria, or both. The antimicrobial agent can, for example, include an antimicrobial peptide, an antibiotic, or both. For example, the antimicrobial agent can include at least one functional group selected from an amine (e.g. a primary amine), an ester, and an acid functional group. Non-limiting examples of antimicrobial peptides include magainins such as magainin 1 and magainin 2. Non-limiting examples of antibiotics include gentamicin, vancomycin, cefotaxime, and the like.

According to another aspect, the present technology relates to a process for producing a transcutaneous intraosseous device as herein defined, the process comprising the following steps:
immobilizing at least one adhesion or proliferation agent onto the surface of the transcutaneous part; and
immobilizing at least one antimicrobial agent onto the surface of the transcutaneous part.

In some examples, the process further includes polishing the surface of the transcutaneous part to decrease the surface roughness prior to the immobilizing steps. For instance, decreasing the surface roughness can promote cell attachment or decrease microbial adhesion. For example, the polishing step can be performed by mechanical polishing, for example, using 240, 600 and/or 1200 grit abrasive papers.

In some examples, the process further includes cleaning the surface of the transcutaneous part prior to the immobilizing steps, and if present, after the polishing step.

For example, the cleaning step can be performed using at least one solvent and/or at least one mild detergent and/or sodium hydroxide (NaOH). For example, the cleaning can be performed by rinsing the surface with the at least one solvent and/or the at least one mild detergent. The cleaning step can, for example, be performed using an ultrasonic bath. For example, cleaning the surface can remove or substantially reduce contaminants, residues (e.g. organic residues), or impurities that are bound to or that have settled on the surface.

In some examples, the immobilization can be non-specific immobilization (i.e., random orientation) or specific immobilization (i.e., uniform orientation). Any type of compatible immobilization is contemplated. Examples of immobilization approaches include, without limitation, covalent immobilization approaches, non-covalent immobilization approaches, immobilization on self-assembled monolayers, or electrochemical methods (e.g. cathodic polarization and anodic polarization). According to one variant of interest, immobilizing the at least one adhesion or proliferation agent and/or antimicrobial agent onto the surface of the transcutaneous part can, independently in each occurrence, involve a covalent approach or a non-covalent approach.

In examples where the immobilization includes a non-covalent approach, said non-covalent approach can be adsorption (e.g. via noncovalent adsorption or physisorption). The adsorption can be carried out by simply immersing the surface into the appropriate solution. For example, the adsorption can be based on weak interactions such as electrostatic forces, hydrogen bonding, Van der Waals forces, or hydrophobic interactions. The binding stability of adsorbed species is controlled by environmental conditions such as pH, ionic strength, or protein concentration. It is to be understood that, if these conditions change, adsorbed molecules can desorb from the surface in an uncontrolled manner.

In examples where immobilization includes a covalent approach, said covalent approach can be, for example, a covalent attachment (e.g. covalent bonding and covalent grafting). Immobilization performed using a covalent approach can lead to a substantially increased binding stability compared to immobilization performed using a non-covalent approach.

In some examples, the covalent attachment can involve at least one free surface reactive group and the process further includes generating the free surface reactive group in a surface pre-functionalization or activation step. The step of generating the free surface reactive group can be performed prior to the immobilizing steps, and if present, after polishing and/or cleaning steps. For example, the free surface reactive group can be a non-polar group, a polar group, or a charged group (e.g. a weakly charged group). For example, the free surface reactive group can include a hydrocarbon-containing group, an oxygen-containing group, a nitrogen-containing group, a phosphorous-containing group, or a sulfur-containing group. Non-limiting examples of free surface reactive groups include hydroxyl groups (—OH groups), amine groups (—NH₂), carboxyl groups (—COOH), and thiol groups (—SH). The process can thus further include the pre-functionalization or activation of the surface of the transcutaneous part prior to the immobilizing steps. For example, using a wet-chemistry functionalization process or a plasma functionalization technique. For instance, the pre-functionalization or activation step includes increasing the number hydroxyl groups (—OH groups) on the surface. For example, pre-functionalization or activation step can be performed by pre-functionalizing or activating the surface with an activating agent such as sodium hydroxide (NaOH), nitric acid (HNO₃), water, plasma, or a piranha solution. The pre-functionalization or activation step can be performed by immersing the surface in the activating agent. For example, the pre-functionalization or activation step can be performed using an ultrasonic bath.

In some examples, the immobilization includes directly immobilizing the least one adhesion or proliferation agent and/or the at least one antimicrobial agent onto the surface of the transcutaneous part. When the least one adhesion or proliferation agent and/or the at least one antimicrobial agent are proteins or peptides, they comprise an N-terminal end (i.e., a free amine functional group (—NH₂) and a C-terminal end (i.e., a free carboxyl functional group (—COOH)) and can further comprise other functional groups. These functional groups can be activated and used to graft the least one adhesion or proliferation agent and/or the at least one antimicrobial agent onto the surface of the transcutaneous part. Examples of functional groups, amino acids comprising them, and surface functional groups which can be required for the covalent immobilization are presented in Table 2.

TABLE 2
Functional groups, amino acids comprising them, and surface
functional groups which can be required for the covalent
immobilization.

| Functional | | Surface functional |
|---|---|---|
| group | Amino acids | groups |
| —NH₂ | Lysine (Lys) | Carboxylic acid |
| | | Active ester |
| | | Aldehyde |
| —SH | Cysteine (Cys) | Maleimide |
| | | Vinyl sulfone |
| | | Amine |
| —COOH | Aspartic acid (Asp) | Amine |
| | Glutamic acid (Glu) | |
| —OH | Serine (Ser) | Epoxy |
| | Threonine (Thr) | |

In some examples, the immobilization can involve at least one bifunctional molecule and the process further includes functionalizing the surface with the at least one bifunctional molecule. The at least one bifunctional molecule comprises identical or different reactive groups on either end of a spacer arm (i.e., homo-bifunctional molecules or hetero-bifunctional molecules). For example, the at least one bifunctional molecule is of formula X₁—R—X₂, where R represents the spacer arm and X₁ and X₂ represent reactive groups. The spacer arm can be an alkyl chain, for example, an alkyl chain having from 10 to 18 carbon atoms, and, in some implementations, from 11 to 16 carbon atoms. According to one variant of interest, the spacer arm is an alkyl chain comprising 16 carbon atoms.

The at least one bifunctional molecule can act as a coupling agent between the surface and the least one adhesion or proliferation agent and/or the at least one antimicrobial agent to be covalently attached. For instance, the at least one bifunctional molecule is intended to keep a fixed distance between the surface and the least one adhesion or proliferation agent and/or the at least one antimicrobial agent to be covalently attached. For instance, the at least one bifunctional molecule can substantially negate steric hindrance, optimized spatial arrangement, or allow a better accessibility of the least one adhesion or proliferation agent and/or the at least one antimicrobial agent for the cells. Any compatible bifunctional molecule is contemplated, for example, the at least one bifunctional molecule can be glutaric anhydride, cis-aconitic anhydride, dopamine, polydopamine, phosphonate-containing bifunctional molecules, and the like.

Non-limiting examples of bifunctional molecules, their functional group, and the functional group of the at least one adhesion or proliferation agent and/or the at least one antimicrobial agent, which can react with the functional group of the bifunctional molecule are presented in Table 3.

TABLE 3
Bifunctional molecules, their functional group, and the
functional group of the least one adhesion or
proliferation agent and/or the at least one
antimicrobial agent which can react with
the functional group of the bifunctional
molecule.

| | | | Functional |
|---|---|---|---|
| | | | group of |
| | | | the least one |
| | Functional | | adhesion or |
| | group | | proliferation agent |
| | of the | | and/or the at least |
| Bifunctional | bifunctional | Bifunctional | one antimicrobial |
| molecule | molecule | reagent | agent |
| Phosphonate- | —COOH | — | Amine groups |
| containing | | | |
| bifunctional | | | |
| molecules | | | |
| Phosphonate- | —NH₂ | — | Acid groups |
| containing | | | Ester groups |
| bifunctional | | | |
| molecules | | | |
| Dopamine or | —NH₂ | Glutaric | Amine groups |
| polydopamine | | anhydride | |

In at least one example, the at least one bifunctional molecule is a phosphonate-containing bifunctional molecule of formula X₁—R—PO(OH)₂, where R is an alkyl as defined herein, and X₁ is a COOH-terminal function or a —NH₂-terminal function allowing the covalent grafting of the least one adhesion or proliferation agent and/or of the at least one antimicrobial agent. The phosphonate-containing bifunctional molecule can also covalently bind to the surface of the transcutaneous part via a reaction with a hydroxyl function.

In at least one example, the at least one bifunctional molecule is dopamine or polydopamine. For example, dopamine or polydopamine can covalently bind to the surface of the transcutaneous part via a reaction with a hydroxyl function. The amine group of dopamine or polydopamine can bind to glutaric anhydride following a ring-opening reaction of glutaric anhydride. The carboxylic acid group of glutaric anhydride, once activated, can react with a free amine of the least one adhesion or proliferation agent and/or the at least one antimicrobial agent. Thus, in this at least one example, the process includes functionalizing the surface with both dopamine or polydopamine and glutaric anhydride.

In some examples, the process further includes activating the at least one bifunctional molecule. For example, activating the at least one bifunctional molecule can be performed by using a cross-linking agent to couple a carboxyl or a phosphate group to a primary amine. In one variant of interest, the cross-linking agent is 1-ethyl-3-(3-dimethylaminopropyl)-carbodiimide (EDC).

### EXAMPLES

The following non-limiting examples are illustrative and should not be construed as limiting the scope of the present invention. These examples will be better understood with reference to the accompanying figures.

### Example 1

### Immobilization of Fibronectin on Ti 6Al-4V ELI

(a) Immobilization of fibronectin on Ti 6Al-4V ELI by adsorption (Ads-FN)

This example illustrates the immobilization of fibronectin on Ti 6Al-4V ELI by adsorption.

Ti 6Al-4V ELI discs (10 mm diameter) were polished to obtain a surface roughness of about 0.2 μm, cleaned, and activated with NaOH to increase the number of hydroxyl groups on the surface of the Ti 6Al-4V ELI discs. Fibronectin (3 μg/mL in 4-(2-hydroxyethyl)-1-piperazineethanesulfonic acid (HEPES), pH 7.4) was adsorbed onto the surface of the Ti 6Al-4V ELI discs.
(b) Immobilization of fibronectin on Ti 6Al-4V ELI by covalent grafting using phosphonate-containing bifunctional molecules

This example illustrates the immobilization of fibronectin on Ti 6Al-4V ELI by covalent grafting using phosphonate-containing bifunctional molecules.

Ti 6Al-4V ELI discs (10 mm diameter) were polished to obtain a surface roughness of about 0.2 μm, cleaned, and activated with NaOH to increase the number of hydroxyl groups on the surface of the Ti 6Al-4V ELI discs. Fibronectin (3 μg/mL in HEPES, pH 7.4) was grafted onto the surface of the Ti 6Al-4V ELI discs. The grafting was carried out using a carboxyl-terminated alkyl phosphonate as a bifunctional molecule having a (—COOH) end group. The —COOH group was activated using a carbodiimide activating moiety and was further reacted with lysine an amino acid of fibronectin.
(c) Immobilization of fibronectin on Ti 6Al-4V ELI by covalent grafting using dopamine and glutaric anhydride as bifunctional molecules (Dop-FN)

This example illustrates the immobilization of fibronectin on Ti 6Al-4V ELI by covalent grafting using dopamine and glutaric anhydride as bifunctional molecules.

Ti 6Al-4V ELI discs (10 mm diameter) were polished to obtain a surface roughness of about 0.2 μm, cleaned and activated with NaOH to increase the number of hydroxyl groups on the surface of the Ti 6Al-4V ELI discs. Fibronectin (3 μg/mL in HEPES, pH 7.4) was grafted onto the surface of the Ti 6Al-4V ELI discs.

The grafting was performed using dopamine and glutaric anhydride as bifunctional molecules. The —COOH group was activated by means of a carbodiimide activating moiety and was further reacted with the lysine amino acids of fibronectin.

(d) Characterization of fibronectin-modified Ti 6Al-4V ELI

This example illustrates the characterization of fibronectin-modified Ti 6Al-4V ELI as described in Examples 1 (a) to (c).

The grafting efficiency of the bifunctional molecule and subsequent fibronectin conjugation onto the surface were evaluated by X-ray photoelectron spectroscopy (XPS). The XPS results are presented in Table 4. As can be seen in Table 4, nitrogen, which can arise from the protein chemical structure and can be associated to the presence of fibronectin, is present for all fibronectin-modified Ti 6Al-4V ELI discs and is not present for the uncoated Ti 6Al-4V ELI disc. As can also be observed in Table 4, the amount of carbon is 31.6% for uncoated Ti 6Al-4V ELI, 40.2% for Ads-FN, 70.9% for Dop-FN, and 62.3% for Phos-FN.

TABLE 4
Surface composition determined by XPS.

| | Composition (%) | | | | |
|---|---|---|---|---|---|
| | C | O | Ti | P | N |
| uncoated Ti | 31.6 | 41.9 | 15.8 | — | — |
| 6Al—4V ELI | | | | | |
| Ad-FN | 40.2 | 44.7 | 11.6 | — | 2.9 |
| Dop-FN | 70.9 | 19.9 | — | — | 8.6 |
| Phos-FN | 62.3 | 26.1 | 5.6 | 3 | 3.9 |

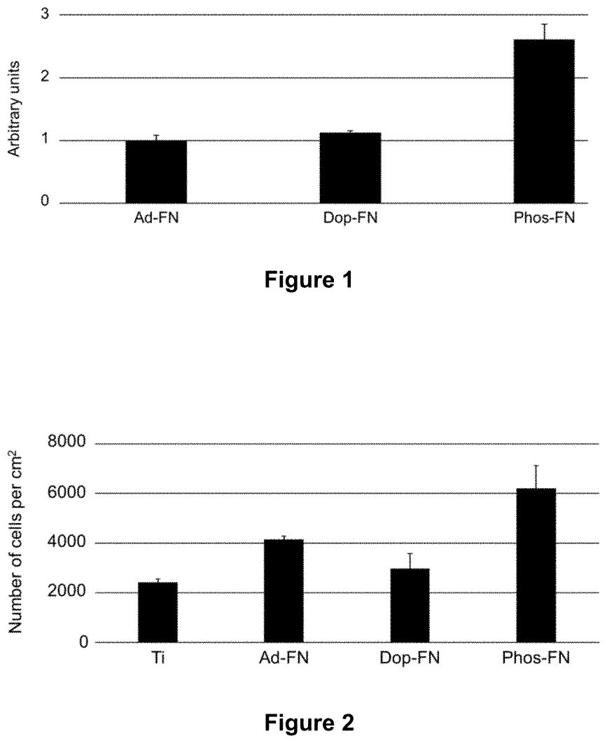

The surface density of adsorbed fibronectin, as well as the availability of fibronectin cell-binding site (RGD) were investigated by ELISA. The availability rate of the cell-binding site (RGD) of immobilized fibronectin-modified Ti 6Al-4V ELI are presented in FIG. 1.

As shown in FIG. 1, the ELISA showed an important change in cell binding site availability (RGD), depending on the protein conjugation strategies. ELISA results evidenced that Phos-FN surfaces exhibited the highest availability of RGD sequences. Phos-FN displayed available cell binding sites that were about 1.5-fold greater than Ad-FN and Dop-FN surfaces.

Cell adhesion assays were also performed. Human dermal fibroblasts passage 4 (HDFs, P4), were seeded on the surface of untreated and fibronectin-modified Ti 6Al-4V ELI discs, at a density of 104 cell/cm². After 24 hours of cell culture, cells were fixed, stained for nuclei with 4′,6-diamidino-2-phenylindole (DAPI) solution and counted under a fluorescent microscope. The cell adhesion assay results are presented in FIG. 2.

As presented in FIG. 2, the biological data showed an increase in the number of cells when comparing the surface of fibronectin-modified Ti 6Al-4V ELI discs to the surface of an untreated Ti 6Al-4V ELI discs. The Phos-FN exhibited significantly improved cell adhesion (Phos-FN>FN-Ads>Dop-FN).

Therefore, these data suggest that the conjugation strategy can affect the spatial conformation of fibronectin, which in turn, can influence the availability of cell binding sites and subsequently, cell adhesion.

### Example 2

### Immobilization of Peptides (RGD or YIGSR Peptides) on Ti 6Al-4V ELI

(a) Immobilization of RGD on Ti 6Al-4V ELI by covalent grafting using phosphonates-containing bifunctional molecules

This example illustrates the immobilization of RGD on Ti 6Al-4V ELI by covalent grafting using phosphonate-containing bifunctional molecules.

Ti 6Al-4V ELI discs were polished with a 1200 grit abrasive paper, then cleaned and activated using NaOH (2.5 M). RGD peptides were covalently grafted on the surface of the Ti 6Al-4V ELI discs using carboxyl-terminated alkane phosphonates. Activated Ti—OH surfaces were reacted in an ethanolic solution containing the carboxyl-terminated alkane phosphonates (1 mM), leading to terminal carboxyl moieties. These moieties were converted into activated esters upon reaction with EDC in 2-(N-morpholino)ethanesulfonic acid (MES) buffer, which, in turn, was used to covalently immobilize RGDS peptides (2×⁻⁵ M) in phosphate-buffered saline (PBS) buffer.
(b) Immobilization of YIGSR on Ti 6Al-4V ELI by covalent grafting using phosphonate-containing bifunctional molecules

This example illustrates the immobilization of YIGSR on Ti 6Al-4V ELI by covalent grafting using phosphonate-containing bifunctional molecules.

Ti 6Al-4V ELI discs were polished with a 1200 grit abrasive paper, then cleaned, and activated using NaOH (2.5 M). YIGSR peptides were covalently grafted on the surface of the Ti 6Al-4V ELI discs using carboxyl-terminated alkane phosphonates. Activated Ti—OH surfaces were reacted in an ethanolic solution containing the carboxyl-terminated alkane phosphonates (1 mM), leading to terminal carboxyl moieties. These moieties were converted into activated esters upon reaction with 1 EDC in MES buffer, which, in turn, was used to covalently immobilize YIGSR peptides (2×10⁻⁵ M) in PBS buffer.
(c) Characterization of the RGD or YIGSR peptides-modified Ti 6Al-4V ELI

This example illustrates the characterization of the peptide-modified Ti 6Al-4V ELI as described in Examples 2 (a) and (b).

The grafting efficiency of the bifunctional molecule and subsequent fibronectin conjugation onto the surface were determined by XPS. The XPS results are presented in Table 5. As can be seen in Table 5, the XPS spectra of alkyl phosphonate self-assembled monolayer films showed a surface concentration of 4% of phosphorus, indicating the presence of phosphonate molecules on the surface. In addition, the presence of nitrogen was also detected after grafting the peptide, thus confirming the presence of the peptides. This result was further confirmed through fluorescence imaging of KRGDS-fluorescein isothiocyanate (FITC) grafted Ti 6Al-4V ELI surfaces.

TABLE 5
Surface composition determined by XPS.

| | Composition (%) | | | | |
|---|---|---|---|---|---|
| | C | O | Ti | P | N |
| Ti—OH | 25.0 ± 0.2 | 53.0 ± 0.4 | 20.0 ± 0.2 | — | 0.4 ± 0.4 |
| Ti—P | 61 ± 3 | 27 ± 2 | 6 ± 1 | 4.0 ± 0.4 | 0.4 ± 0.4 |
| Ti RGDS | 56 ± 1 | 31.9 ± 0.8 | 7.0 ± 0.2 | 2.4 ± 0.8 | 2.7 ± 0.7 |
| Ti YIGSR | 54 ± 3 | 32 ± 2 | 7.9 ± 0.1 | 2.0 ± 0.2 | 3.8 ± 0.9 |

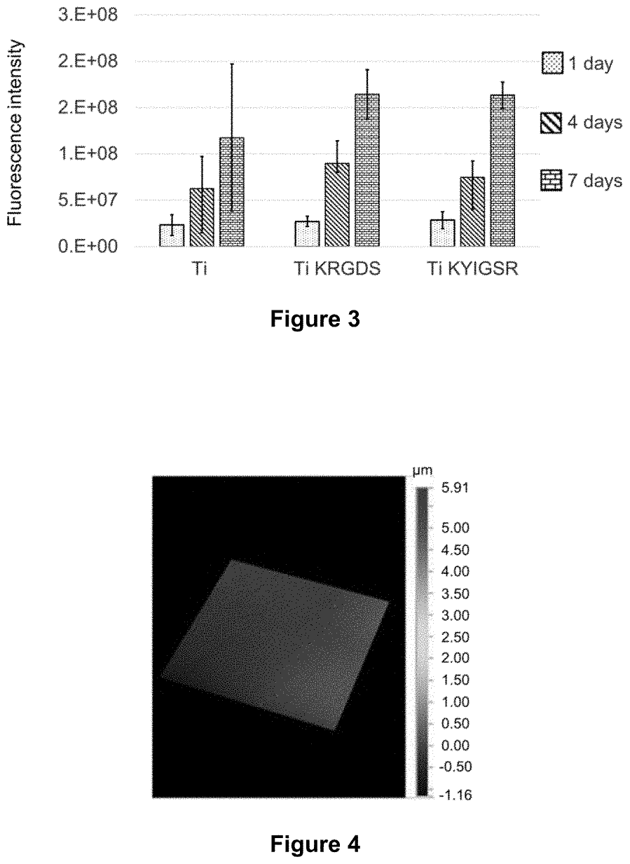

Peptides-grafted Ti 6Al-4V ELI discs were evaluated for their potential to increase skin fibroblasts adhesion and proliferation. After seeding dermal fibroblast cells (10000 cells/cm²) on the surfaces, their attachment, proliferation, and viability were assessed after 1, 4, and 7 days of incubation using 7-hydroxy-3H-phenoxazin-3-one 10-oxide (resazurin) viability assays and immunofluorescence staining. The resazurin assay results are presented in FIG. 3.

As shown in FIG. 3, resazurin assays showed a better skin fibroblasts adhesion and proliferation on RGDS and YIGSR grafted Ti 6Al-4V ELI discs as compared to the uncoated Ti 6Al-4V ELI disc. In addition, after 7 days, the fibroblast viability was significantly improved on grafted Ti 6Al-4V ELI discs.

The results suggested that RGDS and YIGSR peptides were efficiently grafted onto Ti 6Al-4V ELI discs using phosphonate-containing bifunctional molecules. Biological assays exhibited a significant improvement of the skin fibroblasts adhesion, proliferation, and viability on RGDS-modified and YIGSR-modified Ti 6Al-4V ELI discs compared to the uncoated Ti 6Al-4V ELI disc.

### Example 3

### Immobilization of Both Fibronectin and Cefotaxime on Ti 6Al-4V ELI

(a) Immobilization of both fibronectin and cefotaxime on Ti 6Al-4V ELI by covalent grafting using phosphonate-containing bifunctional molecules

This example illustrates the immobilization of both fibronectin and cefotaxime on Ti 6Al-4V ELI discs by covalent grafting using phosphonate-containing bifunctional molecules.

One example of the grafting can be illustrated as in Scheme 1:

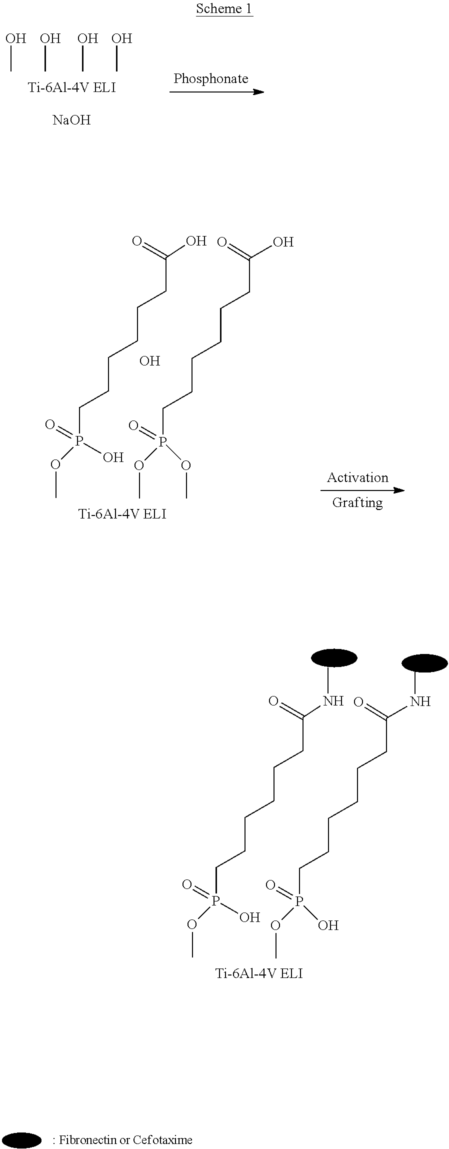

Ti 6Al-4V ELI discs having a thickness of 3 mm and a diameter of 10 mm were polished with a 1200 grit abrasive paper for about 10 minutes to obtain a surface roughness of about 0.2 μm. The Ti 6Al-4V ELI discs were then rinsed with a mild detergent and water and then activated in NaOH (2.5 M) for 15 minutes in an ultrasonic bath to remove contaminants and to increase the number of hydroxyl functions.

After the cleaning step and NaOH activation, the Ti 6Al-4V ELI discs were functionalized using phosphonate-containing bifunctional molecules (1 mM in isopropanol for 24 hours at room temperature and then 24 hours at a temperature of 80° C. under vacuum. The Ti 6Al-4V ELI discs were then activated using 2 mg/ml of EDC in 0.1 M MES for 30 minutes at room temperature before the grafting of fibronectin and cefotaxime. The Ti 6Al-4V ELI discs were rinsed 5 times in HEPES and 3 times in water (vortex). Fibronectin was grafted on the surface by immersing the Ti 6Al-4V ELI discs in 3 mg/ml fibronectin in 10 mM HEPES for 3 hours at room temperature. Cefotaxime was grafted on the Ti 6Al-4V ELI discs by immersing the surface in 50 mg/ml cefotaxime in 10 mM trisaminomethane (Tris) for 3 hours at room temperature.

The Ti 6Al-4V ELI discs were then rinsed five times in HEPES (vortex), 5 times for 2 minutes in HEPES and 0.1% Tween™ 20 in an ultrasonic bath and 3 times in water (vortex) to eliminate adsorption and to ensure that fibronectin and cefotaxime were grafted and not adsorbed.
(b) Immobilization of both fibronectin and cefotaxime on Ti 6Al-4V ELI by covalent grafting using dopamine and glutaric anhydride as bifunctional molecules

This example illustrates the immobilization of both fibronectin and cefotaxime on Ti 6Al-4V ELI by covalent grafting using dopamine and glutaric anhydride as bifunctional molecules.

One example of the grafting can be illustrated as in Scheme 2:

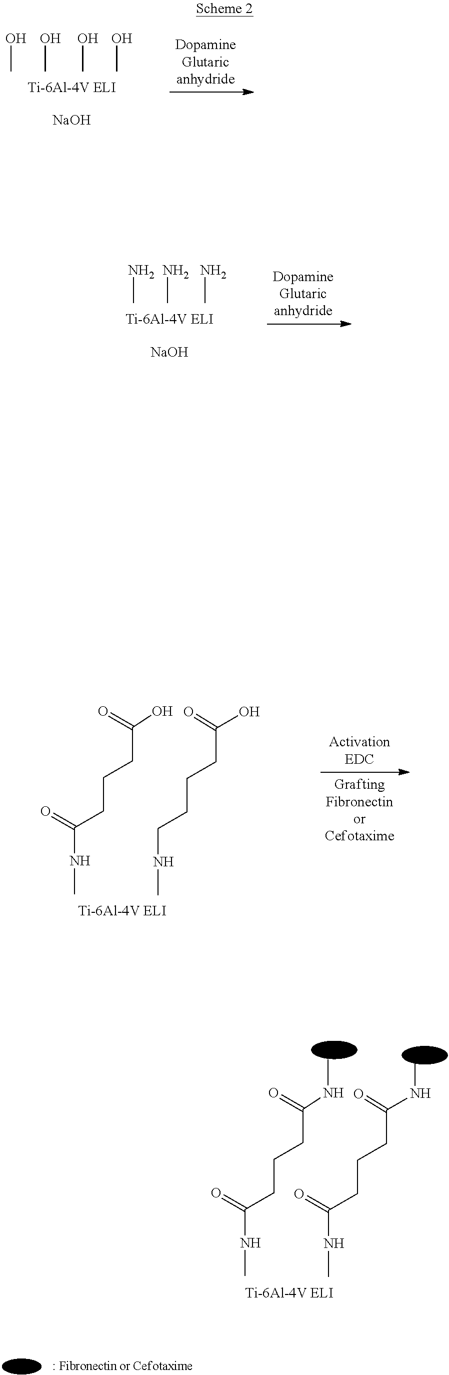

Ti 6Al-4V ELI discs having a thickness of 3 mm and a diameter of 10 mm were polished with a 1200 grit abrasive paper for about 10 minutes to obtain a surface roughness of about 0.2 μm. The Ti 6Al-4V ELI discs were then rinsed with a mild detergent and water and activated in NaOH (2.5 M) for 15 minutes in an ultrasonic bath to remove contaminants and to increase the number of hydroxyl functions.

After the cleaning and the NaOH activation steps, the Ti 6Al-4V ELI discs were functionalized using dopamine and glutaric anhydride as bifunctional molecules (dopamine 2 mg/ml in Tris for 6 hours at room temperature; and then glutaric anhydride 0.1 g/ml). The Ti 6Al-4V ELI discs were then activated using 2 mg/ml of EDC in 0.1M MES for 30 minutes at room temperature before the grafting of fibronectin and cefotaxime. The Ti 6Al-4V ELI discs were rinsed 5 times in HEPES and 3 times in water (vortex). Fibronectin was grafted on the surface by immersing the surface in 3 mg/ml fibronectin in 10 mM HEPES for 3 hours at room temperature. Cefotaxime was grafted on the Ti 6Al-4V ELI discs by immersing the Ti 6Al-4V ELI discs in 50 mg/ml cefotaxime in 10 mM Tris for 3 hours at room temperature.

The Ti 6Al-4V ELI discs were then rinsed five times in the HEPES (vortex), 5 times for 2 minutes in HEPES and 0.1% Tween™ 20 in an ultrasonic bath and 3 times in water (vortex) to eliminate adsorption and to ensure that fibronectin and cefotaxime were grafted and not adsorbed.
(c) Characterization of fibronectin and cefotaxime-modified Ti 6Al-4V ELI

This example illustrates the characterization of fibronectin and cefotaxime-modified Ti 6Al-4V ELI as described in Examples 3 (a) and (b).

The surface roughness of the Ti 6Al-4V ELI discs after the polishing step was characterized. The surface roughness measurements were performed using a Bruker Dektak XT™ contact profilometer. A 5 microns stylus was moved vertically in contact with a sample and then moved laterally across the sample at a stylus tracking force of 0.3 milligrams. A profilometry image obtained for a Ti 6Al-4V ELI disc polished with a 1200 grit abrasive paper is presented in FIG. 4.

As can be seen in FIG. 4, the profilometry image shows that the Ti 6Al-4V ELI disc as prepared in Examples 3 (a) and (b) has a surface roughness of about 0.2 μm.

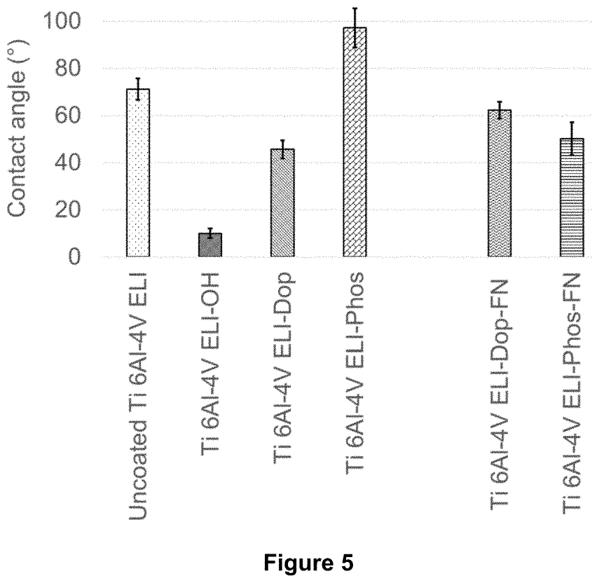

The hydrophilic or hydrophobic character of the functionalized Ti 6Al-4V ELI discs were determined to confirm their modifications after each step. The hydrophilic or hydrophobic character of the functionalized Ti 6Al-4V ELI discs were characterized by measuring the contact angle. These measurements were carried out using a contact angle video system, VCA-2500XE (AST products, Inc., Billerica, Mass., USA), in static mode with a 3 μl drop of nanopure water. Three measurements were carried out on each material and at least two materials were analyzed for each step of the surface modification. The contact angle results obtained after each of the surface modification steps are presented in FIG. 5. FIG. 5 shows the results for uncoated Ti 6Al-4V ELI discs, Ti 6Al-4V ELI discs cleaned and activated by NaOH (Ti—OH), Ti 6Al-4V ELI discs functionalized with phosphonate bifunctional molecules (Ti-Phos), Ti 6Al-4V ELI discs functionalized with dopamine and glutaric anhydride as bifunctional molecules (Ti-Dop), Ti 6Al-4V ELI discs functionalized with dopamine and glutaric anhydride as bifunctional molecules and grafted with fibronectin (Ti-Dop-FN) and Ti 6Al-4V ELI discs functionalized with phosphonate bifunctional molecules and grafted with fibronectin (Ti-Phos-FN).

The chemical treatment of the surfaces with NaOH of the uncoated Ti 6Al-4V ELI discs lead to a reduction of the contact angle from 71°±5° (uncoated Ti 6Al-4V ELI discs) to 20°±2° (Ti—OH). This can be attributed to the removal of hydrophobic organic contaminants and the hydroxylation of the surface, rendering it hydrophilic. When dopamine was grafted, the contact angle increased to 46°±4°. Likewise, after the phosphonate (P16C) was grafted, the 16 carbon atoms alkyl chains rendered the surface hydrophobic (97°±8°. Following the grafting of fibronectin, the contact angle was substantially identical on both surfaces Ti-Dop-FN (62°±4° and Ti-Phos-FN (50°±7°. These results show a different wettability after each of the grafting steps, which indicates the surface modification by the molecules.

Classic XPS analysis (overflight spectrum+high definition spectra) was performed to obtain the atomic composition of the surfaces (overflight spectrum), which was supplemented by a high-resolution analysis of 1s carbon (C1s), oxygen 1s (O1s), and titanium (Ti2p), to obtain information on the chemical environment of the atoms in question.

Each step of modifying or functionalizing the titanium surfaces was characterized by XPS using a PHI 5600-ci spectrometer (Physical Electronics, Minn., USA). The detector opening was at about 4 and the analyzed surface area was about 0.64 mm². For overflight experiments, a standard 300 W Al anode was used, without a neutraliser, at 45° and with a scan range of 1400-0 eV. Three measurements were made on each sample. For the high-resolution experiment, a standard 300 W Mg anode was used, without a neutralizer and at 45°. The characteristic peaks were calculated from a reference at 285.0 eV.

Classic XPS analysis (overflight spectrum+high definition spectra) made it possible to validate the grafting efficiency of dopamine (Ti-Dop-FN) and phosphonate (Ti-Phos-FN) bifunctional molecules. The XPS results of Ti-Dop-FN are presented in Table 6.

TABLE 6
Surface composition determined by XPS after each step for
fibronectin graft with dopamine and GA.

| Surface | Composition (%) | | | |
|---|---|---|---|---|
| treatment | C | O | Ti | N |
| Ti—OH | 21.9 ± 0.8 | 54.8 ± 0.7 | 23 ± 2 | — |
| Ti-Dop | 75 ± 1 | 17.9 ± 0.4 | — | 7.3 ± 0.3 |
| Ti-Dop-GA | 74.7 ± 0.5 | 19.5 ± 0.4 | — | 7 ± 1 |
| Ti-Dop-GA- | 69 ± 1 | 18.9 ± 0.6 | — | 12 ± 1 |
| FN slightly | | | | |
| rinsed | | | | |
| Ti-Dop-GA- | 69 ± 1 | 20.0 ± 0.4 | — | 11 ± 1 |
| FN rinsed | | | | |
| with | | | | |
| Tween ™ 20 | | | | |

As shown in Table 6, the XPS results showed that after the dopamine grafting step, there was no titanium detection on the surface of the Ti 6Al-4V ELI discs. Thus, the polydopamine was grafted homogeneously on the surface of the Ti 6Al-4V ELI discs with an atomic percentage similar to the theoretical atomic composition of dopamine (72.7% C, 18.2% O, and 9.1% N). Fibronectin grafting was confirmed by an increase in the atomic percentage of nitrogen (12% N). In addition, a non-significant difference can be observed between the soft cleaning and the rinsing performed with Tween™ 20. It can therefore be understood that the majority of fibronectin was grafted efficiently on the Ti 6Al-4V ELI discs.

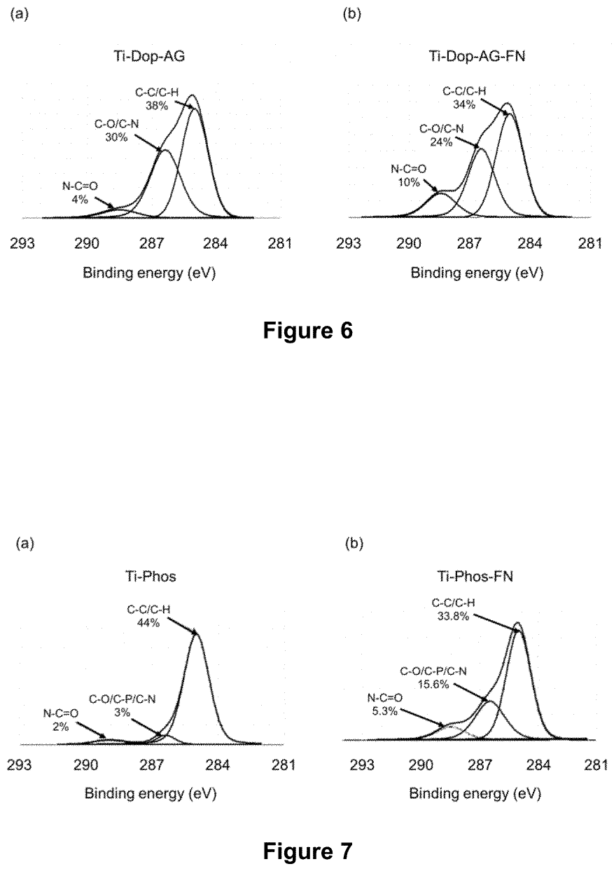

FIG. 6 displays high-resolution XPS scan spectra in (a) for Ti-Dop-GA; and in (b) for Ti-Dop-GA-FN. As shown in FIG. 6, the high resolution XPS spectra of the carbon region (C1s) have three main components. The peaks were deconvoluted and labelled. A 285.0 eV component that represents the C—C and C—H bonds, a C—O and C—N bonds at 286.4 eV, and a 288.4 eV component that represents the N—C═O were observed. As can be seen on FIG. 6 (b), after fibronectin grafting, the peak at 288.4 eV, characteristic of N—C═O bonding increases considerably on the high-resolution C1s spectrum. This component confirms the presence of peptide bonds present in fibronectin.

The XPS results of Ti-Phos-FN are presented in Table 7.

TABLE 7
Surface composition determined by XPS after each step for fibronectin
grafting with phosphonate-containing bifunctional molecules

| Surface | Composition (%) | | | | |
|---|---|---|---|---|---|
| treatment | C | O | Ti | P | N |
| Ti-Phos | 4 ± 2 | 37 ± 2 | 11.0 ± 0.4 | 3.0 ± 0.1 | — |
| Ti-Phos- | 55 ± 1 | 29 ± 0.4 | 7 ± 1 | 2 ± 1 | 8.3 ± 0.2 |
| FN slightly | | | | | |
| rinsed | | | | | |
| Ti-Phos- | 53 ± 0.5 | 29.7 ± 0.4 | 7.0 ± 0.6 | 1.8 ± 0.4 | 8.0 ± 0.3 |
| FN rinsed | | | | | |
| with | | | | | |
| Tween 20 ™ | | | | | |

As shown in Table 7, the XPS results showed that after the phosphonate grafting, the carbon content increased strongly, accompanied by a decrease in the titanium content and an appearance of phosphorus, which proves that phosphonate molecules have been grafted on the surface of the Ti 6Al-4V ELI discs. This can be confirmed by the fact that the ratio P/C (0.06) is equal to the theoretical ratio. After the grafting of fibronectin, a decrease in the titanium content was observed, accompanied by a substantially strong appearance of nitrogen. These results confirm the presence of fibronectin on the surface of the Ti 6Al-4V ELI discs. In addition, after rinsing with Tween 20™, the nitrogen dropped slightly, which can indicate that fibronectin is substantially efficiently grafted onto the surface of the Ti 6Al-4V ELI discs.

FIG. 7 displays high-resolution XPS scan spectra in (a) for Ti-Phos; and in (b) for Ti-Phos-FN. As shown in FIG. 7, the high resolution XPS C1s spectra have three main components. The peaks were deconvoluted and labelled. The bonds C—O , C—P, and C—N as well as N—C═O (amides) increased significantly after fibronectin grafting, since the protein contains a large amount of amino acids that contain these types of bonds.

Chemical derivation was also performed; the primary amines present at the surface of the Ti 6Al-4V ELI discs following dopamine grafting were quantified by chemical vapor phase derivation using 4-(trifluoromethyl)benzaldehyde (TFBA). TFBA can easily be identifiable and quantifiable by XPS due to the presence of three fluorine atoms, which allow the detection of the molecule by XPS on the substrates. Generally, the reaction of TFBA with primary amines can be performed as illustrated in Scheme 3.

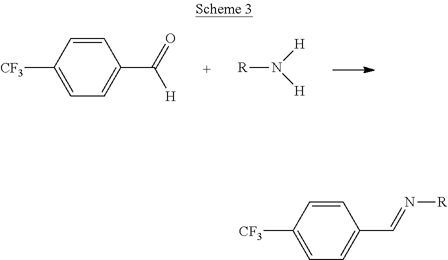

The concentration of amines at the surface of the Ti 6Al-4V ELI discs can be calculated using Equation 1:

$$[ \% {NH}_{2} ] = \frac{[ \% F ]}{300 - 11 [ \% F ]} \times 100 \qquad [ Eq . 1 ]$$

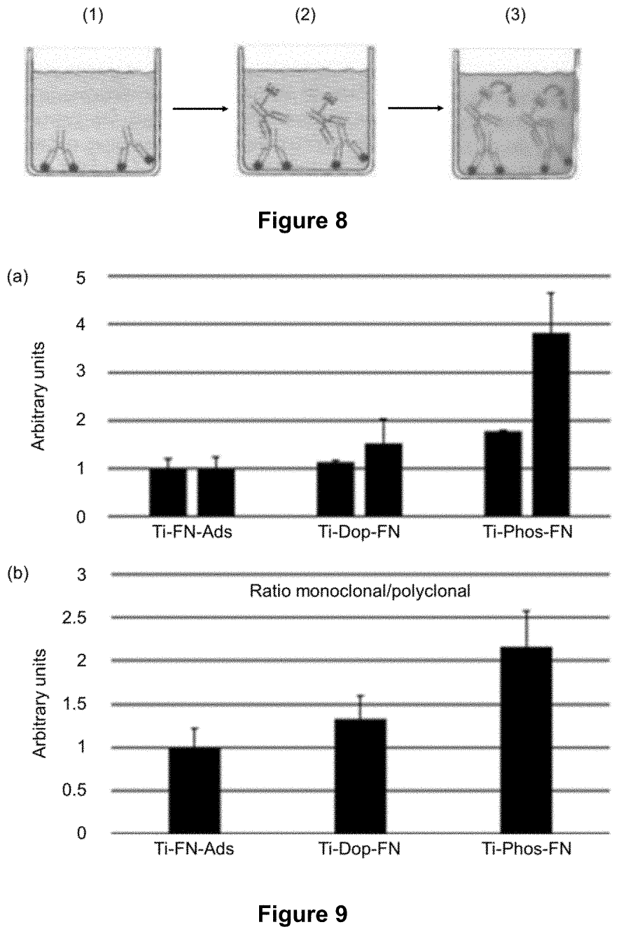

The bioactivity of fibronectin was evaluated by indirect ELISA to detect the presence of fibronectin and fibronectin (RGD) cell adhesion sites on modified Ti 6Al-4V ELI surfaces. This test was carried out in 3 steps as illustrated in FIG. 8.

As shown in FIG. 8, the first step (1) consists in incubating a primary antibody in wells, the primary antibody reacts specifically with fibronectin or with fibronectin cell adhesion fragment (RGD). The second step (2) consists in the incubation of a secondary antibody coupled to a peroxidase, which recognizes the primary antibody. In the third step (3), a substrate (Amplex™ Red reagent+H₂O₂) specific to the incubated enzyme turns pink if the reaction is positive (i.e., in the presence of fibronectin or RGD). The intensity of the staining is proportional to the amount of enzyme present and therefore to the amount of fibronectin or RGD. This intensity can then be measured using a plate reader.

FIG. 9 displays a graph of the ELISA results performed on modified surfaces where fibronectin was adsorbed or grafted via a phosphonate bifunctional molecule or a dopamine bifunctional molecule. FIG. 9 presents the results in (a) for the use of two antibodies, one polyclonal which reacts with fibronectin and the other monoclonal which reacts with RGD cell adhesion sites; and in (b) the evaluation of monoclonal/polyclonal antibodies ratios.

The cells adhere to fibronectin in part through the recognition of the RGD sequence. Thus, the accessibility of this sequence in fibronectin was determined by ELISA. As shown in FIG. 9, the ELISA test carried out with a polyclonal antibody shows a slight increase in the amount of fibronectin on the Ti-Phos-FN when compared to the other two surfaces Ti-FN ads and Ti-Dop-FN.

As can also be seen in FIG. 9 (a), the ELISA test carried out with a monoclonal antibody shows that fibronectin cell adhesion sites (RGD) are more available when grafted by phosphonates than by dopamine or when they are simply adsorbed. This can be further confirmed by the signal ratio of monoclonal fluorescence to polyclonal shown in FIG. 9 (b). Indeed, this ratio describes the availability of RGD cell adhesion sites relative to the total amount of fibronectin at the surface. These results show that the grafting strategy significantly influences the bioactivity of fibronectin and suggests that fibronectin-grafted by phosphonates can favor the attachment of fibroblasts and keratinocytes.

The in-vitro evaluation of the cell adhesion and/or antibacterial effect of the different functionalized Ti 6Al-4V ELI discs also determine the influence of the grafting strategy on the adhesion of fibroblasts and keratinocytes and the effect of antimicrobial agent on the survival, adhesion and proliferation of fibroblasts and keratinocytes.

Fibroblasts or keratinocytes were cultured in treated cell culture flasks, in a basal medium composed of Dulbecco's Modified Eagle Medium (DMEM), 10% fetal calf serum and 1% penicillin/streptomycin. The cells were incubated in a humid atmosphere containing 5% of CO₂ at a temperature of 37° C. The fibroblasts or keratinocytes were seeded on the modified Ti 6Al-4V ELI discs for biological testing.

Cell viability tests were performed with blue trypan using an optical microscope. The number of dead and live cells were counted using a hemocytometer to determine the percentage of viability. Similarly, the modified Ti 6Al-4V ELI discs were treated with resazurin to evaluate the metabolic activity of the cells by measuring their fluorescence using a plate reader. Cell adhesion assays were performed by immunofluorescence labelling of Vinculin to visualize focal contacts of cells on different surfaces after specific incubation times. The cells were also visualized using a scanning electron microscope (SEM) to observe their morphology. For the proliferation assay, the cells were seeded at 10,000 cells per cm² on the modified Ti 6Al-4V ELI discs.

The antibacterial effect of cefotaxime was studied on Escherichia coli and Staphylococcus aureus bacteria with an adhesion assay (i.e., colony count, CFU) and a viability test (membrane integrity, LIVE/DEAD assay). Each sample was immersed in a tube containing the bacterial suspensions. The tubes were then placed under agitation in an incubator for 3 hours. After the incubation period, the adhered bacteria were plated in several Petri dishes. The Petri dishes were then placed in the incubator for 24 hours. The colonies were subsequently counted to determine the colony forming units (CFU) and to calculate the bacterial ratio based on Equation 2.

$$Bacterial ratio = \frac{{CFU}_{control} - {CFU}_{Experimental}}{{CFU}_{control}} \times 100 \qquad [ Eq . 2 ]$$

The remaining bacterial suspension was stained with a mixture of the two dyes for the LIVE/DEAD assay and then visualized under a fluorescence microscope. Depending on the integrity of the cell membrane, they were stained in green (living, intact membrane) or in red (dead, compromised membrane).

### Example 4

### Immobilization of KRGDS or KYIGSR and Magainin 2 on Ti 6Al-4V ELI

(a) Immobilization of KRGDS or KYIGSR and magainin 2 on Ti 6Al-4V ELI by covalent grafting using phosphonate-containing bifunctional molecules

This example illustrates the immobilization of KRGDS or KYIGSR and magainin 2 on Ti 6Al-4V ELI by covalent grafting using phosphonate-containing bifunctional molecules.

Ti 6Al-4V ELI discs having a thickness of 3 mm and a diameter of 9.4 mm were polished with a 1200 grit abrasive paper to obtain a surface roughness of about 0.2 μm. The Ti 6Al-4V ELI discs were then cleaned in an ultrasonic bath with several solvents, and then activated in NaOH (2.5 M) for 15 minutes to remove contaminants and increase the number of hydroxyl functions. After the cleaning step and the NaOH activation step, the Ti 6Al-4V ELI discs were functionalized using phosphonate-containing bifunctional molecules by immersing the discs in a 1 mM phosphonate solution in anhydrous isopropanol and by incubating the discs for 24 hours at room temperature. The discs were then placed in an oven for 24 hours at a temperature of 80° C. The discs were subsequently rinsed with ethanol and dried. The discs were then placed in a 2×10⁻⁵ M HEPES (10 mM, pH=7.45) buffer for 3 hours under stirring. The grafting of the magainin 2 peptides was performed by the same protocol but in a 25 μg/ml magainin 2 solution. The discs were later rinsed 3 times with HEPES and 5 times with water under vortex agitation to remove the peptides adsorbed on the surface. Finally, the discs were air-dried.
(b) Immobilization of KRGDS or KYIGSR and magainin 2 on Ti 6Al-4V ELI by covalent grafting using dopamine and glutaric anhydride as bifunctional molecules

This example illustrates the immobilization of KRGDS or KYIGSR and magainin 2 on Ti 6Al-4V ELI by covalent grafting using dopamine and glutaric anhydride as bifunctional molecules.

Ti 6Al-4V ELI discs having a thickness of 3 mm and a diameter of 9.4 mm were polished with a 1200 grit abrasive paper to obtain a surface roughness of about 0.2 μm. The Ti 6Al-4V ELI discs were then cleaned in an ultrasonic bath with several solvents, and then activated in NaOH (2.5 M) for 15 minutes to substantially remove contaminants and increase the number of hydroxyl functions.

After the cleaning step and NaOH activation, the Ti 6Al-4V ELI discs were functionalized by immersing the discs in a 2 mg/ml dopamine solution in Tris (pH=8.5) in a flask. The flask was then agitated for 6 hours. The discs were then rinsed with ultrapure water under vortex agitation. The discs were subsequently treated with glutaric anhydride, which was then activated using EDC before the grafting of KRGDS or KYIGSR and magainin 2. The discs were then placed in a 2×10⁻⁵ M peptide (KRGDS or KYIGSR) solution in HEPES (10 mM, pH=7.45) buffer for 3 hours under stirring. The grafting of magainin 2 peptides was performed by the same protocol but in a 25 μg/ml magainin 2 solution. The discs were then rinsed 3 times with HEPES and 5 times with water under vortex agitation to remove peptides adsorbed on the surface. Finally, the discs were air-dried.
(c) Characterization of KRGDS or KYIGSR and magainin 2-modified Ti 6Al-4V ELI

This example illustrates the characterization of KRGDS or KYIGSR and magainin 2-modified Ti 6Al-4V ELI as described in Examples 4 (a) and (b).

The KRGDS or KYIGSR and magainin 2-modified Ti 6Al-4V ELI discs were characterized as described in Example 3 (c).

The contact angle results after each step are presented in Table 8.

TABLE 8
Surface composition determined by XPS after each step
for fibronectin grafting with phosphonate-containing
bifunctional molecules

| Treatment conditions/grafting | Contact angle |
|---|---|
| Ti 6Al—4V ELI NaOH | 44 ± 2 |
| Ti 6Al—4V ELI + polydopamine 6 h | 46 ± 4 |
| Ti 6Al—4V ELI + polydopamine 6 h + | 30 ± 7 |
| GA + KRGDS | |
| Ti 6Al—4V ELI + polydopamine 6 h + | 19 ± 2 |
| GA + KYIGSR | |
| Ti 6Al—4V ELI + polydopamine 6 h + | 24 ± 2 |
| GA + margainin 2 | |
| Ti 6Al—4V ELI + Phosphonate | 97 ± 8 |
| Ti 6Al—4V ELI + phosphonate + | 89 ± 3 |
| KRGDS | |
| Ti 6Al—4V ELI + phosphonate + | 67 ± 3 |
| KYIGSR | |
| Ti 6Al—4V ELI + phosphonate + | 73 ± 3 |
| magainin 2 | |

As can be seen in Table 8, the contact angle increased after grafting phosphonate groups, which can be attributed to the alkyl chains of the phosphonates. The contact angle decreases after grafting the peptides KRGDS, KYIGSR and magainin 2, which can be attributed to the presence of the amino groups and the hydroxyl groups in the chemical structure of the grafted peptides. The decrease in the contact angle value confirms that modifications have been made to the surfaces after the grafting of each of the peptides.

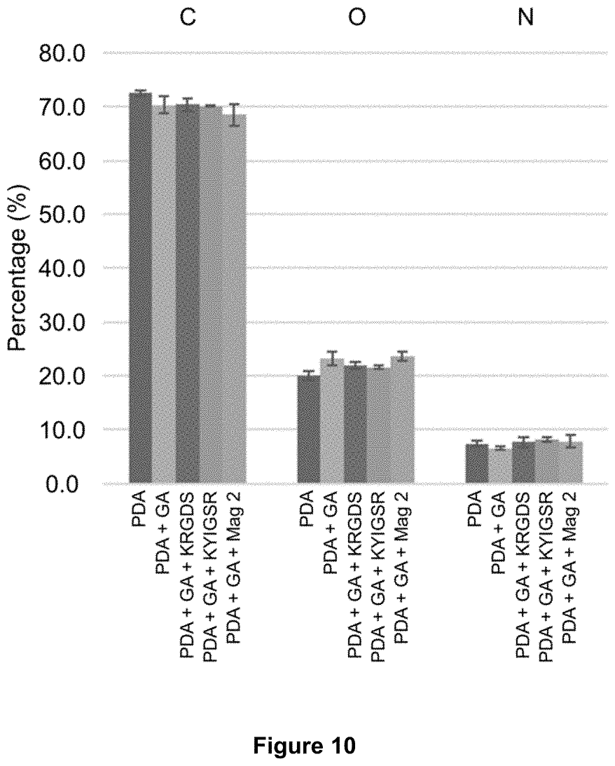

Classic XPS analysis (overflight spectrum +high definition spectra) was also performed. The carbon, oxygen, and nitrogen levels obtained after each of the surface modification steps are presented in FIG. 10, for the discs prepared in Example 4 (b). As shown in FIG. 10, it is difficult to confirm the presence of the peptides when grafted via polydopamine (PDA), since PDA is mainly composed of C, O, and N. The results are presented for PDA, for PDA-GA, for PDA-GA-KRGDS, PDA-GA-KYIGSR, and PDA-GA-Mag 2.

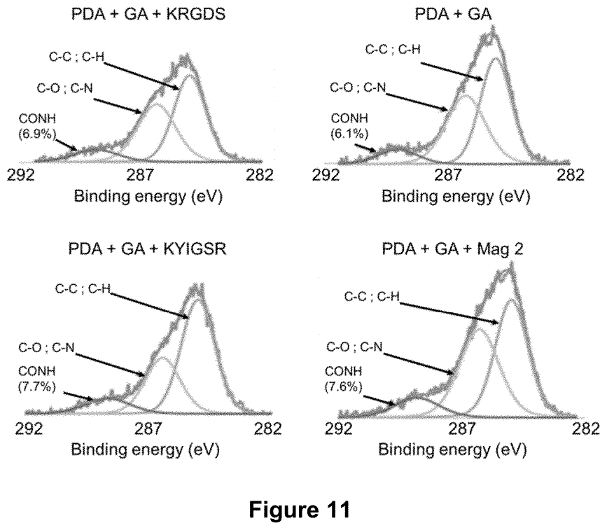

FIG. 11 displays high-resolution XPS spectra for PDA-GA-KRGDS, PDA-GA, PDA-GA-KYIGSR, and PDA-GA-Mag 2. As shown in FIG. 11, the high resolution XPS C1s spectra have three main components. The peaks were deconvoluted and labelled. The results are presented for C—O, and C—N bonds, CONH bonds, and C—C and C—H bonds. The high-resolution XPS thus demonstrate that the area of the third contribution, which corresponds to the peptide bond CONH, is greater after the grafting of the three peptides. This can confirm the presence of the peptides on the surfaces functionalized by dopamine.

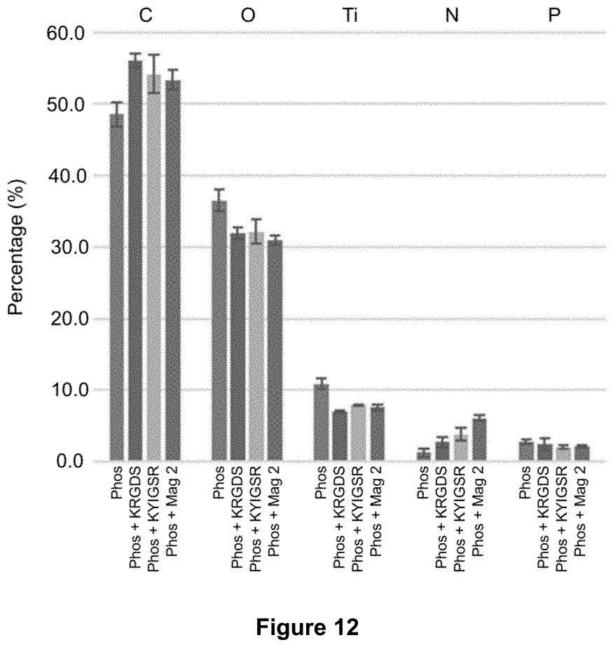

The carbon, oxygen, and nitrogen levels obtained after each of the surface modification steps are presented in FIG. 12, for the discs prepared in Example 4 (b). As shown in FIG. 12, there is an increase in the nitrogen content and a decrease in the titanium content after grafting of the peptides on the surfaces functionalized with the phosphonates. Thus, confirming that the peptides are present on the functionalized surfaces by the phosphonates.

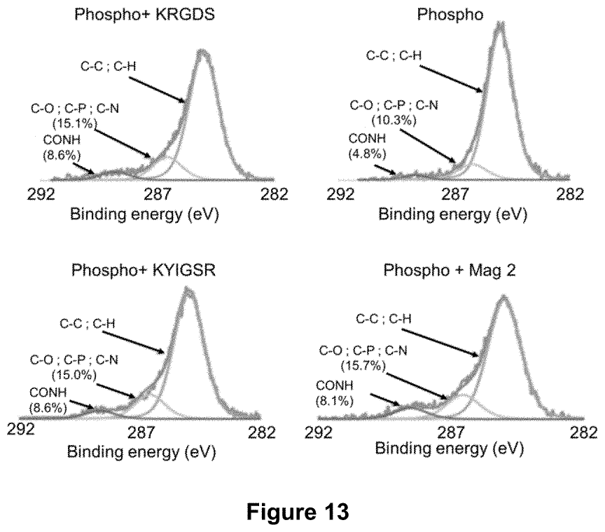

FIG. 13 displays high-resolution XPS scan spectra for Phos-KRGDS, Phos, Phos-KYIGSR, and Phos-Mag 2. As shown in FIG. 13, the high resolution XPS C1s spectra have three main components. The peaks were deconvoluted and labelled. The results are presented for C—O , C—P and C—N bonds, CONH bonds, and for C—C and C—H bonds.

According to FIG. 13, the area of the third contribution, which corresponds to the peptide bond CONH, is greater after the grafting of the three peptides. The area of the second contribution, corresponding to the C—O and C—N bonds, also increases for all peptides.

The presence of peptides on surfaces functionalized with phosphonates was thus confirmed by XPS (increase of the percentage of nitrogen and of groups C—O , C—N and CONH) and by contact angle (more hydrophilic surface).

1. A transcutaneous intraosseous device comprising an intraosseous part and a transcutaneous part, wherein at least a surface of said transcutaneous part is provided with at least one adhesion or proliferation agent and at least one antimicrobial agent.

2. The transcutaneous intraosseous device of claim 1, wherein said transcutaneous intraosseous device comprises an intraosseous transcutaneous implant used to anchor a prosthetic limb or a dental prothesis.

3. The transcutaneous intraosseous device of claim 1, wherein the surface of said transcutaneous part has a roughness of less than about 1.1 μm.

4. The transcutaneous intraosseous device of claim 1, wherein said transcutaneous intraosseous device comprises at least one biomaterial and the at least one biomaterial is at least one of: a metal and a metal alloy.

5. The transcutaneous intraosseous device of claim 1, wherein:
the at least one adhesion or proliferation agent comprises an extracellular macromolecule selected from the group consisting of a protein and an adhesion protein extracted from the extracellular matrix; or
the at least one adhesion or proliferation agent comprises a cell adhesion peptide extracted from the extracellular matrix.

6. The transcutaneous intraosseous device of claim 1, wherein the at least one antimicrobial agent comprises at least one of an antimicrobial peptide and an antibiotic.

7. An intraosseous transcutaneous amputation prosthesis (ITAP) comprising an intraosseous part and a transcutaneous part, wherein at least a surface of said transcutaneous part is provided with at least one adhesion or proliferation agent and at least one antimicrobial agent.

8. The ITAP of claim 7, wherein the surface of said transcutaneous part has a roughness of less than about 1.1 μm.

9. The ITAP of claim 7, wherein said ITAP comprises at least one stainless steel-based, titanium-based, or cobalt-based biomaterial.

10. The ITAP of claim 7, wherein:
the at least one adhesion or proliferation agent comprises an adhesion protein selected from fibronectin and laminin; or
the at least one adhesion or proliferation agent comprises a cell adhesion peptide and said cell adhesion peptide comprises at least one of RGD, KRGD, YIGSR and KYIGSR.

11. The ITAP of claim 7, wherein the at least one antimicrobial agent comprises at least one of an antimicrobial peptide and an antibiotic, and said antimicrobial peptide is a magainin selected from magainin 1 and magainin 2, and said antibiotic comprises at least one of gentamicin, vancomycin, and cefotaxime.

12. A process for producing a transcutaneous intraosseous device as defined in claim 1, comprising the following steps:
immobilizing at least one adhesion or proliferation agent onto the surface of the transcutaneous part; and
immobilizing at least one antimicrobial agent onto the surface of the transcutaneous part.

13. The process of claim 12, wherein immobilizing the at least one adhesion or proliferation agent and the at least one antimicrobial agent, independently in each occurrence, involves a covalent approach or a non-covalent approach.

14. The process of claim 12, wherein immobilizing the at least one adhesion or proliferation agent and the at least one antimicrobial agent is performed directly on the surface.

15. The process of claim 12, further comprising at least one of the following steps:
polishing the surface of the transcutaneous part prior to the immobilizing steps, wherein said polishing is performed by mechanical polishing using abrasive papers;
cleaning the surface of the transcutaneous part prior to the immobilizing steps, and if present, after the polishing step, wherein the cleaning is carried out in an ultrasonic bath and/or using at least one of solvent and a mild detergent;
pre-functionalizing or activating the surface prior to the immobilizing steps;
immobilizing at least one bifunctional molecule onto the surface of the transcutaneous part, and
if present, activating said at least one bifunctional molecule, wherein the activating step is performed by using a cross-linking agent.

16. The process of claim 15, wherein the surface pre-functionalization or activation step comprises generating at least one free surface reactive group comprising at least one of a hydrocarbon-containing group, an oxygen-containing group, a nitrogen-containing group, a phosphorous-containing group, and a sulfur-containing group or at least one of a hydroxyl group (—OH), an amine group (—NH₂), a carboxyl group (—COOH), and a thiol group (—SH).

17. The process of claim 15, wherein the surface pre-functionalization or activation step is carried out using an activating agent and is performed by at least one of a wet-chemistry functionalization process and a plasma functionalization technique, wherein the activating agent comprises at least one of a sodium hydroxide solution, a nitric acid solution, and a piranha solution.

18. The process of claim 15, wherein the at least one bifunctional molecule comprises identical or different reactive groups on a first end and a second end of a spacer arm, said spacer arm being an alkyl chain comprising from 10 to 18 carbon atoms, and wherein said at least one bifunctional molecule comprises at least one of glutaric anhydride, cis-aconitic anhydride, dopamine, polydopamine, and a phosphonate-containing bifunctional molecule.

19. The process of claim 18, wherein immobilizing the at least one bifunctional molecule is performed by at least one of:covalently binding the first end of the space arm onto the surface and by covalently binding the second end of the space arm to the at least one adhesion or proliferation agent and the at least one antimicrobial agent.

20. The process of claim 19, wherein the at least one bifunctional molecule is a first bifunctional molecule and the process further comprises:
immobilizing at least one second bifunctional molecule by covalently binding a second end of a space arm to at least one of the at least one second bifunctional molecule, to the at least one adhesion or proliferation agent and to the at least to one antimicrobial agent, covalently binding a first end of the spacer arm of the at least one second bifunctional molecule to the second end of the spacer arm of the at least one first bifunctional molecule and covalently binding the first end of the spacer arm of the at least one first bifunctional molecule onto the surface; and
optionally activating the at least one second bifunctional molecule, wherein the activating step is performed by using a cross-linking agent.

21. The process of claim 20, wherein the at least one first bifunctional molecule comprises dopamine or polydopamine and the at least one second bifunctional molecule comprises glutaric anhydride.
